(19) United States
(12) Patent Application Publication
(10) Pub. No.: US 2007/0104785 A1
(43) Pub. Date: May 10, 2007

(54) TABLETS OF LINEZOLID FORM III AND PROCESSES FOR THEIR PREPARATION

(76) Inventors: Suryakant Vamanrao Navale, Gurgaon (IN); Rahul S. Dabre, Gurgaon (IN); Ajay Kumar Singla, Chandigarh (IN); Tarun Vijan, Panchkula (IN)

Correspondence Address: RANBAXY INC., 600 COLLEGE ROAD EAST, SUITE 2100, PRINCETON, NJ, 08540, US

(21) Appl. No.: 11/460,825

(22) Filed: Jul. 28, 2006

(30) Foreign Application Priority Data
Jul. 29, 2005 (IN) 2018/DEL/2005

(51) Int. Cl. A61K 9/46; A61K 31/5377

(52) U.S. Cl. 424/466; 514/235.2

(57) ABSTRACT

The present invention relates to solid oral dosage forms of linezolid polymorphic Form III with reproducible dissolution profile and processes for their preparation. The solid dosage form includes linezolid Form III, one or more of means to reduce the gelling tendency of linezolid form III, and one or more of pharmaceutically acceptable excipients.

### TECHNICAL FIELD OF THE INVENTION

The present invention relates to solid oral dosage forms of linezolid polymorphic form III with reproducible dissolution profile and processes for their preparation.

### BACKGROUND OF THE INVENTION

Linezolid is a well-known synthetic antibacterial agent belonging to the class of oxazolidinone derivatives. Chemically, it is (S)—N—[[3-[3-Fluoro-4-(4-morpholinyl)phenyl]-2-oxo-5-oxazolidinyl]methyl]-acetamide. It is used in the treatment of vancomycin-resistant Enterococcus faecium infections; nosocomial pneumonia; complicated skin and skin structure infections including diabetic foot infections, without concomitant osteomyelitis; uncomplicated skin and skin structure infections and community acquired pneumonia. It is available as 600 mg tablets sold under the brand name of Zyvox®. It is given two to three times a day.

Linezolid is known to exhibit polymorphism and three crystalline forms are known. Linezolid Form I is known from the publication, Brickner S J et al. (J. Med. Chem. 39 (3), 673-679, 1996). U.S. Pat. No. 6,559,305 discloses linezolid Form II. Tablets containing linezolid Form II are known from U.S. Pat. No. 6,514,529. The PCT application WO 05035530 discloses crystalline Form III of linezolid. The crystalline Form III is disclosed as a thermally stable form and the bulk solid is more compact and less electrostatic than Form II and hence may be more readily subjected to any treatment under the usual conditions of the pharmaceutical technology, in particular, of formulation on an industrial scale. Our experiments while designing a solid oral dosage form with linezolid Form III revealed that linezolid Form III exhibits a gelling tendency when the solid dosage form comes in contact with water. The gel formation leads to an erratic dissolution of the linezolid from the dosage form. A dosage form should deliver a drug in a reproducible way so that a maximum therapeutic effect can be achieved. An unpredictable release from the dosage form can compromise efficacy. The gelling tendency of linezolid Form III would discourage its formulation into solid dosage forms.

It is desirable to develop solid dosage forms containing linezolid Form III and which provide reproducible dissolution profiles comparable to a reference standard. We hereby disclose solid dosage forms comprising linezolid Form III that provide reproducible dissolution profiles in an aqueous media and processes for the preparation of these solid dosage forms.

### SUMMARY OF THE INVENTION

In one general aspect, there is provided a solid dosage form comprising linezolid Form III and one or more of means to reduce the gelling tendency of linezolid Form III and one or more pharmaceutically acceptable excipients.

In another aspect, there is provided a solid dosage form of linezolid Form III comprising a means to reduce a gelling tendency of linezolid Form III and one or more pharmaceutically acceptable excipients, wherein the solid dosage form provides reproducible dissolution of linezolid in an aqueous media.

In another general aspect, there is provided a solid dosage form of linezolid Form III comprising means to reduce the gelling tendency of linezolid Form III and one or more pharmaceutically acceptable excipients, wherein the solid dosage form releases not less than 90% of linezolid in 60 minutes in an aqueous media.

In another aspect, there is provided a solid dosage form of linezolid Form III comprising a carbon dioxide source to reduce the gelling tendency of linezolid Form III and one or more pharmaceutically acceptable excipients.

In another aspect, there is provided a solid dosage form of linezolid Form III comprising a water-insoluble polymer to reduce the gelling tendency of linezolid Form III and one or more pharmaceutically acceptable excipients.

In another aspect, there is provided a solid dosage form of linezolid Form III comprising colloidal silicon dioxide to reduce the gelling tendency of linezolid Form III and one or more pharmaceutically acceptable excipients.

In another aspect, there is provided a solid dosage form of linezolid Form III comprising a pharmaceutically acceptable clay to reduce the gelling tendency of linezolid Form III and one or more pharmaceutically acceptable excipients.

In another aspect, there is provided a process for the preparation of solid dosage form of linezolid Form III, wherein the process comprises contacting linezolid Form III with one or more of gelling reducing means and optionally one or more of diluent(s), binder(s), disintegrant(s), glidant(s), lubricant(s) and processing the mixture into a solid dosage form.

In another aspect, there is provided a method of treating a medical condition in a human responsive to linezolid therapy wherein the method comprises administering a solid dosage form comprising linezolid Form III, a gelling reducing means and one or more pharmaceutically acceptable excipients.

The details of one or more embodiments of the inventions are set forth in the description below. Other features, objects and advantages of the inventions will be apparent from the description and claims.

### DETAILED DESCRIPTION OF THE INVENTION

Linezolid Form III can be produced by the process set forth in International Publication WO 05035530. Linezolid Form III is obtained by heating linezolid in a known crystalline form or in a mixture of known crystalline forms until the known forms are converted to Form III. The known form may be heated directly to obtain linezolid Form III; or linezolid Form III may be obtained by heating linezolid suspended in a solvent such as toluene, xylene, etc.

The term “aqueous media” herein refers to water, simulated gastric and intestinal fluids as well as gastric and intestinal fluids found in the gastrointestinal tract of the human body.

Without wishing to be bound by any theory, the following explanation is given for mechanisms involved with the solid dosage form of the present invention that provide a reproducible dissolution profile. Linezolid Form III has an acceptable solubility of about 3 mg/mL in water. When linezolid Form III crystal comes in contact with water, the surface of the crystal dissolves and the dissolved linezolid Form III instead of spreading in water tends to stick to the crystal surface to form a gel layer around the crystal. The gel formation around the crystal acts as a barrier impeding further contact with water, which in turn retards the solubilization of linezolid Form III. The formation of gel, if stopped, would lead to rapid, predictable dissolution of linezolid Form III in aqueous media.

We have found that a solid oral dosage form comprising 600 mg of linezolid Form III and containing the means to disrupt or retard the gel formation provides dissolution of linezolid comparable to that of the reference 600 mg linezolid tablet sold under the trade name Zyvox® marketed by Pharmacia and Upjohn.

The “means to reduce gelling tendency” or “gelling reducing means” may be selected from a wide range of pharmaceutical excipients. For example, an effervescent couple which can produce gas bubbles when in contact with water can help disrupt the gel layer around the linezolid Form III crystal. A substance that can produce effervescence when in contact with gastric acid would also help overcome the formation of gel layer. Another means of overcoming gelation would be to avoid a rapid solvation of linezolid Form III crystals with water by mixing or coating linezolid Form III with a water insoluble polymer. The slow solvation would make the gel layer thin, if at all, if the gel layer is formed. The other means would be mixing or coating the linezolid Form III with pharmaceutically acceptable clays and colloidal silicon dioxide which prevent linezolid Form III particle aggregation. The combination of these means is also envisaged in the present dosage forms.

The term “contacting” means mixing linezolid Form III with one or more of gelling reducing means or coating linezolid Form III with one or more of gelling reducing means. The contacting may be achieved by conventional methods known to those skilled in the art. Linezolid Form III may also be mixed with a gelling reducing means after granulation with one or more excipients.

The term “carbon dioxide source” as used herein refers to substance or substances which when coming in contact with aqueous media generate carbon dioxide with effervescence. The carbon dioxide source may be a combination of a base component and an acid component commonly known as an effervescent couple. Effervescent couples react in the presence of water to generate carbon dioxide. Usually, the base component comprises an alkali metal or alkaline earth metal carbonate or bicarbonate and the acid component comprises an aliphatic carboxylic acid. The alkali metal or alkaline earth metal carbonate or bicarbonate may be selected from sodium carbonate, potassium carbonate, sodium bicarbonate, potassium bicarbonate, calcium bicarbonate, magnesium carbonate and mixtures thereof. The acid component may be selected from the group consisting of citric acid, tartaric acid, malic acid, fumaric acid, adipic acid, acid citrates, succinic acid and mixtures thereof. The base component can also be used alone as it would react with gastric acid to generate carbon dioxide gas. The effervescent couple may comprise from about 5% to about 30% by weight of the solid dosage form. The base component if used alone may comprise from about 5% to about 20% by weight of the solid dosage form.

The water-insoluble polymer may be selected from cellulose derivatives (e.g. ethylcellulose), polyvinyl acetate, neutral copolymers based on ethyl acrylate and methylmethacrylate, copolymers of acrylic and methacrylic acid esters with quaternary ammonium groups, such as Eudragit NE, RS or RS30D, RL or RL30D and the like. The water insoluble polymer may comprise from about 1.0% to about 10% by weight of the solid dosage form.

Clays may be selected from calcium silicate, magnesium silicate, magnesium trisilicate and the like and may comprise from about 1% to about 10% by weight of the solid dosage form. Colloidal silicon dioxide may be that which is commonly available such as sold under the brand name Aerosil® and may comprise from about 1% to about 10% by weight of the solid dosage form.

The amount of linezolid Form III in the solid dosage form may be up to about 90% by weight of the tablet, particularly up to about 80% and more particularly up to about 70% by weight of the tablet.

Besides the above ingredients the solid dosage form may further comprise other pharmaceutical excipients, such as one or more of diluent(s), binder(s), disintegrant(s), glidant(s) and lubricant(s).

The one or more diluents may be selected from mannitol, sorbitol, xylitol, lactose, microcrystalline cellulose, magnesium carbonate, calcium carbonate, dicalcium phosphate, tribasic calcium phosphate, calcium sulphate, and the like. The diluent may comprise from about 10% to about 40% by weight of the solid dosage form.

The one or more binders may be selected from polyvinylpyrrolidone; hydroxypropyl cellulose, hydroxypropyl methylcellulose and other such materials routinely used in the art of solid dosage forms. The binder may be present in an amount varying from about 1% to about 10% by weight of the solid dosage form.

The one or more disintegrants may be selected from cross-linked carboxymethylcellulose and its sodium salt, crospovidone, sodium starch glycolate, sodium carboxymethyl cellulose, calcium carboxymethyl cellulose, low-substituted hydroxypropyl cellulose and sodium alginate or any other such material routinely used in the art of solid dosage forms. The disintegrant may be added intragranulary or extragranularly or both. The concentration of the disintegrant may vary from 1% to 15% by weight of the solid dosage form.

The glidant may be talc.

The one or more lubricants may be selected from the group consisting of magnesium stearate, zinc stearate, calcium stearate, sodium stearyl fumarate, stearic acid, and the like. The lubricant may be used in a concentration varying from 0.5% to 2.5 % by weight of the tablet.

The solid dosage form may be selected from tablets, granules, capsules, sachet containing the granules, and powders and capsules filled with granules or powder. A particularly suitable solid dosage form is the tablet. The tablets can be prepared by dry granulation and spray granulation methods.

Dry granulation can be performed by roller compaction or slugging. The blend comprising linezolid Form III, one or more of means to reduce the gelling tendency of linezolid Form III, and one or more of other pharmaceutical excipients is roller compacted into a compact, such as a sheet or ribbon, which is typically milled to produce granules. The granules so obtained may be optionally mixed with one or more pharmaceutical excipients and compressed into a tablet or filled into a capsule. Alternative to roller compaction, the above blend may be slugged by means of a tablet press. The slug that is formed is milled into granules, which in turn may be recompressed into tablet or filled into capsule. Alternatively, the means may also be added extragranularly.

Spray granulation may be done in a fluid bed granulator. The blend comprising linezolid Form III, one or more of other pharmaceutical excipients selected from diluent and disintegrant may be spray granulated with a dispersion of water insoluble polymer or colloidal silicone dioxide; the granules dried, optionally mixed with one or more pharmaceutical excipients, and compressed into a tablet or filled into a capsule. Alternatively, the blend comprising linezolid Form III, one or more of other pharmaceutical excipient selected from diluent and disintegrant may be granulated with a binder solution and mixed with one or more of gelling reducing means and other pharmaceutical excipients and compressed into a tablet or filled into a capsule.

Tablets may be coated with coating compositions such as Opadry® sold by Colorcon to impart aesthetic appeal. Such a coating may comprise about 3% by weight of the tablet.

In one embodiment, the tablet of linezolid form III may be prepared by mixing linezolid Form III, a carbon dioxide source, diluent, disintegrant and binder and compacting the blend using a roller compactor; sizing the compacts into granules; mixing with diluent and lubricant, and compressing into a tablet using appropriate tooling.

In another embodiment, the tablet of linezolid Form III may be prepared by granulating a mixture of linezolid Form III, diluent, binder with an aqueous dispersion of a water insoluble polymer optionally containing a plasticizer and a water soluble polymer; drying and mixing the granules with one or more of diluent, disintegrant, glidant and lubricant, and compressing into a tablet using appropriate tooling.

In another embodiment, the tablet of linezolid Form III may be prepared by spray granulating a mixture of linezolid Form III and diluent with an aqueous dispersion of colloidal silicon dioxide and subsequently with a binder solution; drying and mixing the granules with one or more of diluent, disintegrant, glidant and lubricant and compressing into a tablet using appropriate tooling.

In another embodiment, the tablet of linezolid Form III may be prepared by spray granulating a mixture of linezolid Form III, diluent and colloidal silicon dioxide with a binder solution; drying and mixing the granules with one or more of diluent, disintegrant, glidant and lubricant and compressing into a tablet using appropriate tooling.

In another embodiment, the tablet of linezolid Form III may be prepared by spray granulating a mixture of linezolid Form III, diluent, with aqueous dispersion of pharmaceutically acceptable clay and subsequently with a binder solution; drying and mixing the granules with one or more of diluent, disintegrant, glidant and lubricant and compressing into a tablet using appropriate tooling.

In another embodiment, the tablet of linezolid Form III may be prepared by spray granulating a mixture of linezolid Form III, diluent and pharmaceutically acceptable clay with a binder solution; drying and mixing the granules with one or more of diluent, disintegrant, glidant and lubricant and compressing into a tablet using appropriate tooling.

The following examples are illustrative of the invention, and are not intended to be construed as limiting the invention.

### Comparative Example A

| | | Qty. |
|---|---|---|
| S. No. | INGREDIENTS | (mg/tab) |
| | Intragranular | |
| 1 | Linezolid (Form III) | 600.00 |
| 2 | Microcrystalline Cellulose | 84.0 |
| 3 | Hydroxypropyl cellulose | 12.00 |
| 4 | Purified water | q.s. |
| | Extra granular | |
| 5 | Microcrystalline Cellulose | 93.6 |
| 6 | Sodium starch glycolate | 42.0 |
| 7 | Magnesium Stearate | 8.4 |
| | Core tablet | 840.00 |

Procedure: Linezolid, microcrystalline cellulose and part of hydroxypropylcellulose are granulated with an aqueous solution of the remaining part of hydroxypropylcellulose in a rapid mixer granulator. The granules are dried in a fluid bed dryer and mixed with extragranular microcrystalline cellulose, sodium starch glycolate and the final blend is lubricated with magnesium stearate, and compressed into tablets using appropriate tooling.

### Comparative Example B

| | | Qty. |
|---|---|---|
| S. No. | INGREDIENTS | (mg/tab) |
| | Intragranular | |
| 1 | Linezolid (form III) | 600.00 |
| 2 | Magnesium stearate | 3.00 |
| | Extra granular | |
| 3 | Microcrystalline Cellulose | 444.00 |
| 4 | Crospovidone | 35.00 |
| 5 | Sodium lauryl sulfate | 10.0 |
| 6 | Magnesium Stearate | 8.0 |
| | Core tablet | 1100.00 |

Procedure: Linezolid and magnesium stearate are blended and compacted on a roller compactor. The compacts are sifted on an oscillating granulator to form granules. Microcrystalline cellulose, crospovidone and sodium lauryl sulphate are blended and the blend mixed with the granules formed above. The final blend is lubricated with magnesium stearate and compressed into tablets using appropriate tooling.

### Example 1

| | | Qty. |
|---|---|---|
| S. No. | INGREDIENTS | (mg/tab) |
| | Intragranular | |
| 1 | Linezolid (form III) | 600.00 |
| 2 | Microcrystalline Cellulose | 160.00 |
| 3 | Sodium Bicarbonate | 100.00 |
| 4 | Citric acid anhydrous | 100.00 |
| 5 | Crospovidone | 25.00 |
| 6 | Sodium lauryl sulfate | 25.00 |
| 7 | Colloidal silicon dioxide | 20.00 |
| 8 | Sodium chloride | 10.0 |
| 9 | Magnesium stearate | 5.00 |
| | Extra granular | |
| 10 | Microcrystalline Cellulose | 100.00 |
| 11 | Magnesium stearate | 5.00 |
| | Core tablet | 1150.00 |

Process: Linezolid, microcrystalline cellulose, sodium bicarbonate, citric acid anhydrous, sodium lauryl sulfate, colloidal silicon dioxide and sodium chloride are sifted through a suitable mesh and lubricated with magnesium stearate by mixing in a suitable blender for five minutes. The blend is compacted with a roller compactor to prepare the compacts. The compacts are sized into granules using an oscillating granulator and blended with sifted microcrystalline cellulose. The final blend is lubricated with magnesium stearate and compressed into tablets using appropriate tooling.

### Example 2

Qty. (mg/tab)

| S. No. | INGREDIENTS | 2 (i) | 2 (ii) |
|---|---|---|---|
| | Intragranular | | |
| 1 | Linezolid (form III) | 600 | 600 |
| 2 | Microcrystalline cellulose | 180 | 180 |
| 3 | Hydroxypropylmethyl cellulose | 20 | 50 |
| 4 | Ethyl cellulose | 78 | 50 |
| 5 | Triethyl citrate | 7.8 | 7.8 |
| 6 | P. water | q.s. | q.s. |
| | Extragranular | | |
| 7 | Microcrystalline cellulose | 137.2 | 135.2 |
| 8 | Crosscarmellose sodium | 55 | 55 |
| 9 | Colloidal silicon dioxide | 11 | 11 |
| 10 | Magnesium stearate | 11 | 11 |
| | Total | 1100 | 1100 |

Process: Linezolid and microcrystalline cellulose are sifted through a suitable mesh, mixed in a blender and spray granulated using mixture of ethyl cellulose aqueous dispersion, triethyl citrate and hydroxypropyl methylcellulose in water in a fluid bed processor. The granules are dried, sifted through a suitable mesh and mixed with extragranular microcrystalline cellulose, crosscarmellose sodium and colloidal silicon dioxide. The blend is lubricated with magnesium stearate and compressed into a tablet using appropriate tooling.

### Example 3:

| | | Qty. |
|---|---|---|
| S. No. | INGREDIENTS | (mg/tab) |
| | Intragranular | |
| 1 | Linezolid (form III) | 600.00 |
| 2 | Microcrystalline Cellulose | 180.00 |
| 3 | Colloidal silicon dioxide | 50.00 |
| 4 | Hydroxypropyl methylcellulose | 33.00 |
| 5 | Purified water | q.s. |
| | Extragranular | |
| 6 | Silicified microcrystalline Cellulose | 160.0 |
| 7 | Croscarmellose Sodium | 55.00 |
| 8 | Talc | 11.00 |
| 9 | Magnesium Stearate | 11.00 |
| | Core tablet | 1100.0 |

Process: Linezolid and microcrystalline cellulose are sifted through a suitable mesh and the blend is spray granulated with aqueous dispersion of colloidal silicon dioxide followed by a solution of hydroxypropyl methylcellulose in water in a fluid bed processor. The resulting granules are dried, sifted through a suitable mesh and mixed with extragranular silicified microcrystalline cellulose, crosscarmellose sodium and colloidal silicon dioxide. The blend is lubricated with magnesium stearate and talc and compressed into tablets using appropriate tooling. The tablets are coated with a coating composition of Opadry to a weight gain of 3% by weight of the core tablet.

### Example 4

| | | Qty. |
|---|---|---|
| S. No. | INGREDIENTS | (mg/tab) |
| | Intragranular | |
| 1 | Linezolid (form III) | 600.00 |
| 2 | Microcrystalline Cellulose | 300.00 |
| 3 | Povidone | 50.00 |
| 4 | Sodium lauryl sulfate | 20.00 |
| 5 | Purified water | q.s. |
| | Extragranular | |
| 6 | Microcrystalline Cellulose | 18.00 |
| 7 | Croscarmellose Sodium | 40.00 |
| 8 | Sodium bicarbonate anhydrous | 100.00 |
| 9 | Colloidal Silicon dioxide | 11.00 |
| 10 | Magnesium Stearate | +UZ,25/301.00 |
| | Core tablet | 1150.00 |

Process: Linezolid and microcrystalline cellulose are sifted through a suitable mesh, blended and spray granulated using a solution of povidone and sodium lauryl sulfate in water in fluid bed processor. The resulting dried granules are sifted through a suitable mesh, and mixed with extragranular microcrystalline cellulose, croscarmellose sodium, sodium bicarbonate and colloidal silicon dioxide. The blend is lubricated with magnesium stearate and compressed into a tablet using appropriate tooling. The tablet is coated with a coating composition of Opadry to a weight gain of 3% by weight of the core tablet.

TABLE 1
Dissolution profiles for comparative Examples A and B and Examples
1-4 in 900 mL of 0.01N HCl in USP apparatus II at 50 rpm.

| | % Drug dissolved | | | | | | | |
|---|---|---|---|---|---|---|---|---|
| | Reference Standard | | | | | | | |
| Time | (Zyvox; Linezolid | Comparative | Comparative | | Example 2 | | | |
| in min | tablets 600 mg) | Example A | Example B | Example 1 | 2 (i) | 2 (ii) | Example 3 | Example 4 |
| 10 | 91.0 | 66.9 | 38.7 | 90.8 | 84.7 | 64.8 | 91.8 | 93.8 |
| 20 | 96.3 | 72.1 | — | — | 94.5 | 98.0 | 94.5 | — |
| 30 | 95.5 | 76.0 | 48.7 | 92.4 | 97.8 | 101.4 | 96.1 | 96.8 |
| 60 | 97.7 | 81.9 | 55.5 | 95.3 | 97.7 | 104.8 | — | 99.5 |

The dissolution was almost complete in 60 minutes for the tablets of Examples 1, 2, 3 and 4, containing the means to reduce the gelling tendency of linezolid Form III in comparison with tablets of comparative Examples A and B not containing any means to reduce the gelling tendency of linezolid Form III. Furthermore, the dissolution profiles for the tablets of examples 1, 2, 3 and 4 are comparable to that of the reference standard (Zyvox; Linezolid tablets 600 mg).

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim:

1. A solid dosage form comprising linezolid Form III, one or more of means to reduce the gelling tendency of linezolid form III, and one or more of pharmaceutically acceptable excipients.

2. The solid dosage form as claimed in claim 1, wherein the solid dosage form releases not less than 90% of linezolid in 60 minutes.

3. The solid dosage form as claimed in claim 1, wherein the means to reduce the gelling tendency of linezolid Form III comprises one or more of a carbon dioxide source, water insoluble polymer(s), colloidal silicon dioxide and pharmaceutically acceptable clay(s).

4. The solid dosage form as claimed in claim 3, wherein the carbon dioxide source is selected from an effervescent couple and base component.

5. The solid dosage form as claimed in claim 4, wherein the effervescent couple comprises an acidic component and a base component.

6. The solid dosage form as claimed in claim 5, wherein the acidic component is selected from one or more of citric acid, tartaric acid, malic acid, fumaric acid, adipic acid, acid citrates and succinic acid.

7. The solid dosage form as claimed in claim 4, wherein the base component is selected from one or more of sodium carbonate, potassium carbonate, sodium bicarbonate, potassium bicarbonate, calcium bicarbonate, and magnesium carbonate.

8. The solid dosage form as claimed in claim 3, wherein the water insoluble polymer is selected from one or more of cellulose derivatives, polyvinyl acetate, neutral copolymers based on ethyl acrylate and methylmethacrylate, copolymers of acrylic and methacrylic acid esters with quaternary ammonium groups.

9. The solid dosage form as claimed in claim 3, wherein the pharmaceutically acceptable clay is selected from one or more of calcium silicate, magnesium silicate and magnesium trisilicate.

10. The solid dosage form as claimed in claim 1, wherein the linezolid Form III comprises up to 90% by weight of the tablet.

11. The solid dosage form as claimed in claim 1, wherein the one or more pharmaceutically acceptable excipients are selected from one or more of diluents, binders, disintegrants, and lubricants.

12. The solid dosage form as claimed in claim 11, wherein the diluent is selected from one or more of mannitol, sorbitol, xylitol, lactose, microcrystalline cellulose, magnesium carbonate, calcium carbonate, dicalcium phosphate, tribasic calcium phosphate, and calcium sulphate.

13. The solid dosage form as claimed in claim 11, wherein the binder is selected from one or more of polyvinylpyrrolidone; hydroxypropyl cellulose, and hydroxypropyl methylcellulose.

14. The solid dosage form as claimed in claim 11, wherein the disintegrant is selected from one or more of cross-linked carboxymethylcellulose and its sodium salt, crospovidone, sodium starch glycolate, sodium carboxymethyl cellulose, calcium carboxymethyl cellulose, low-substituted hydroxypropyl cellulose and sodium alginate.

15. The solid dosage form as claimed in claim 11, wherein the lubricant is selected from one or more of magnesium stearate, zinc stearate, calcium stearate, sodium stearyl fumarate, and stearic acid.

16. The solid dosage form as claimed in claim 1, wherein the solid dosage form is selected from tablets, granules, capsules, sachet containing the granules, powders and capsules filled with granules or powder.

17. A process for the preparation of a solid dosage form of linezolid Form III, wherein the process comprises contacting linezolid Form III with one or more of gelling reducing means and optionally one or more of diluent(s), binder(s), disintegrant(s), glidant(s), lubricant(s) and processing into a solid dosage form.

18. The process according to claim 17, wherein the process comprises mixing linezolid Form III with one or more gelling reducing means and optionally one or more of diluent(s), binder(s), disintegrant(s), glidant(s), lubricant(s) and processing the mixture into a solid dosage form.

19. The process according to claim 17, wherein the process comprises coating linezolid Form III with one or more gelling reducing means and optionally mixing with one or more of diluent(s), binder(s), disintegrant(s), glidant(s), lubricant(s) and processing the mixture into a solid dosage form.

20. The process as claimed in claim 17, wherein the solid dosage form comprises a tablet, capsule, granules, powder, sachet containing the granules or powder and capsules filled with granules or powder.

21. The process as claimed in claim 17, wherein the mixture is processed into solid dosage form by wet granulation, dry granulation or direct compression.

22. The process as claimed in claim 20, wherein the mixture is processed into solid dosage form by wet granulation, dry granulation or direct compression.
